(19) United States
(12) Patent Application Publication
(10) Pub. No.: US 2008/0200772 A1
(43) Pub. Date: Aug. 21, 2008

(54) Bionic information measuring system, bionic information measuring transmitter and bionic information measuring receiver

(76) Inventor: Hiroshi Shimizu, Chiba-shi (JP)

Correspondence Address: BRUCE L. ADAMS, ESQ;ADAMS & WILKS, SUITE 1231, 17 BATTERY PLACE, NEW YORK, NY, 10004, US

(21) Appl. No.: 12/012,831

(22) Filed: Feb. 6, 2008

(30) Foreign Application Priority Data
Feb. 19, 2007 (JP) 2007-038279

(51) Int. Cl. A61B 5/00

(52) U.S. Cl. 600/300

(57) ABSTRACT

To reduce power consumption of a receiver in a bionic information measuring system. A transmitter calculates a heart rate per predetermined time period by detecting heartbeat of a measured person and transmits in wireless a heartbeat information signal including heartbeat information and transmission interval information at a transmission interval in accordance with a state of setting a switch and a state of a power source in reference to a state table stored to a memory. A receiver receives the heartbeat information signal from the transmitter at reception timing in synchronism with the transmission interval information included in the heartbeat information signal and displays the heartbeat information included in the heartbeat information signal by a display portion.

### BACKGROUND OF THE INVENTION

1. Field of the Invention

The present invention relates to a bionic information measuring system for transmitting bionic information of a measured person measured on a side of a transmitter to a side of a receiver and carrying out a notice indicating the bionic information on the side of the receiver, a bionic information measuring transmitter and a bionic information measuring receiver used in the bionic information measuring system.

2. Description of the Related Art

In a background art, there has been developed a bionic information measuring system of a portable type for measuring bionic information of a measured person such as heartbeat, pulse, a step number or the like.

For example, according to a heartbeat measuring system constituting a kind of the bionic information measuring system, generally, the system includes a transmitter for detecting heartbeat of a user and transmitting in wireless a heartbeat information signal constituting an information signal with regard to the heartbeat, and a receiver for carrying out a display of a heart rate or the like by receiving the heartbeat information signal from the transmitter and is constituted such that a user carries to use at least one of the transmitter and the receiver.

According to a heartbeat monitoring system described in Patent Reference JP-A-5-168604 (paragraphs [0006] through [0041] and FIG. 1 through FIG. 7), the system is constituted such that a transmitter detects heartbeat by a sensor mounted to the chest portion of a measured person and transmitting in wireless a heartbeat information signal and the heartbeat information signal is received by a receiver of a wristwatch type to display a heart rate or the like. Thereby, the measured person can always be informed of the heart rate or the like of one's own by display of the receiver of the wristwatch type mounted to the arm of one's own.

According to the heartbeat monitoring system described in JP-A-5-168604, the transmitter and the receiver are for portable use, and therefore, a battery is used as a power source. The transmitter is mounted to the chest portion, and therefore, even when a shape thereof is comparatively large, a problem is not posed practically, and therefore, a large-sized battery having a large capacity can be used even when power consumption is considerable.

However, the receiver is a small-sized apparatus of a size of about a wristwatch, and therefore, even when power consumption is considerable, a large-sized battery having a large capacity cannot be used. Therefore, even when the receiver is small-sized and the power consumption is considerable, the small-sized battery having the small capacity is obliged to be used, battery life of the receiver is shorten to pose a problem that interchange of the battery (charge in a case of a secondary battery) needs to be carried out frequently.

The problem stays the same not only in the system of measuring heartbeat but also in a system of measuring various kinds of bionic information such as an energometer or the like.

### SUMMARY OF THE INVENTION

It is an aspect of the present invention to provide a bionic information measuring system reducing power consumption of a receiver therein.

It is another aspect of the present invention to provide a bionic information measuring transmitter and a bionic information measuring receiver suitable for constructing the bionic information measuring system.

According to the invention, there is provided a bionic information measuring system characterized in a bionic information measuring system including a transmitter for detecting a bionic signal of a measured person and transmitting in wireless a bionic information signal including bionic information in correspondence with the bionic signal at predetermined intervals and a receiver for carrying out a notice indicating the bionic information included in the bionic information signal received by the receiver, wherein the transmitter includes sensor means for detecting the bionic signal of the measured person and outputting the bionic information in correspondence therewith, and transmitting means for transmitting a signal constituted by adding transmission interval information indicating a transmission interval of the bionic information to the bionic information as the bionic information signal, and wherein the receiver includes receiving means for receiving the bionic information signal, controlling means for setting a reception interval of the receiving means based on the transmission interval information included in the bionic information signal, and noticing means for carrying out the notice indicating the bionic information of the measured person based on the bionic information signal received by the receiving means.

On the side of the transmitter, the sensor means detects the bionic signal of the measured person and outputs the bionic information in correspondence therewith, the transmitting means transmits the signal constituted by adding the transmission interval information indicating the transmission interval of the bionic information to the bionic information as the bionic information signal. On the side of the receiver, the controlling means sets the reception interval of the receiving means based on the transmission interval information included in the bionic information signal, the receiving means receives the bionic information signal, and the noticing means carries out the notice indicating the bionic information of the measured person based on the bionic information received by the receiving means.

Here, there may be constructed a constitution in which the transmitter includes transmission interval setting means for setting the transmission interval of the bionic information signal, and the transmitting means transmits the bionic information signal by the transmission interval set by the transmission interval setting means.

Further, there may be constructed a constitution in which the transmitter is constituted by including at least one of operating means capable of setting the transmission interval by an external operation and transmitter power source state detecting means for detecting a state of a power source for the transmitter and setting the transmission interval in accordance with the state of the power source as the transmission interval setting means.

Further, there may be constructed a constitution in which in a case of including at least the transmitter power source state detecting means as the transmission interval setting means, when the transmitter power source state detecting means detects that the power source of the transmitter is reduced to be equal to or smaller than a predetermined voltage and sets the transmission interval, the transmission interval is set the most predominantly by the transmitter power source state detecting means.

Further, there may be constructed a constitution in which the transmitting means of the transmitter transmits the bionic information signal by a plurality of kinds of the transmission intervals, wherein the receiver includes reception interval setting means for setting a reception interval of the receiving means, the receiving means receives the bionic information signal transmitted by the transmission interval in synchronism with the reception interval set by the reception interval setting means, and the noticing means carries out the notice indicating the bionic information of the measured person based on the received bionic information signal.

Further, there may be constructed a constitution in which the reception interval setting means is constituted by including at least one of operating means capable of setting the reception interval, the receiver power source state detecting means for detecting a state of a power source of the receiver and setting the reception interval in accordance with the state of the power source, and mode detecting means for detecting an operation mode of counting means provided on a side of the receiver and setting the reception interval in accordance with the operation mode of the counting means.

Further, there may be constructed a constitution in which in a case of including at least the receiver power source state detecting means as the reception interval setting means, when the receiver power source state detecting means detects that the power source for the receiver is reduced to be equal to or smaller than a predetermined voltage and sets the reception interval, the reception interval is set the most predominantly by the receiver power source state detecting means.

Further, according to the invention, there is provided a transmitter for measuring bionic information characterized in a transmitter for measuring bionic information for detecting a bionic signal of a measured person and transmitting in wireless a bionic information signal including bionic information in correspondence with the bionic signal at predetermined intervals, the transmitter including sensor means for detecting the bionic signal of the measured person and outputting the bionic information in correspondence therewith, and transmitting means for transmitting a signal constituted by adding transmission interval information indicating a transmission interval of the bionic signal to the bionic information as the bionic information signal. The sensor means detects the bionic signal of the measured person and outputs the corresponding bionic information, and the transmitting means transmits as the bionic information signal a signal added with the transmission interval information indicating the transmission interval of the bionic information to the bionic information.

Here, there may be constructed a constitution including transmission interval setting means for setting a transmission interval of the bionic information signal, wherein the transmitting means transmits the bionic information signal by the transmission interval set by the transmission interval setting means.

Further, as the transmission interval setting means, there may be constructed a constitution in which the transmission interval setting means includes at least one of an operating means capable of setting the transmission interval by an external operation and transmitter power source state detecting means for detecting a state of a power source for the transmitter and setting the transmission interval in accordance with the state of the power source.

Further, there may be constructed a constitution in which in a case of including at least the transmitter power source state detecting means as the transmission interval setting means, when the transmitter power source state detecting means detects that the power source for the transmitter is reduced to be equal to or smaller than a predetermined voltage and sets the transmission interval, the transmission interval is set the most predominantly by the transmitter power source state detecting means.

Further, there may be constructed a constitution including the transmitting means transmits the bionic information signal by a plurality of kinds of the transmission intervals.

Further, according to the invention, there is provided a receiver for measuring bionic information characterized in a receiver for measuring bionic information for receiving a bionic information signal including bionic information of a measured person and transmission interval information indicating a transmission interval of the bionic information and carrying out a notice indicating the bionic information of the measured person, the receiver including receiving means for receiving the bionic information signal, controlling means for setting a reception interval of the receiving means based on transmission interval information included in the bionic information signal, and noticing means for carrying out a notice indicating the bionic information of the measured person based on the bionic information signal received by the receiving means. The controlling means sets the reception interval of the receiving means based on the transmission interval information included in the bionic information signal, and the noticing means performs the notice indicating the bionic information of the measured person based on the bionic information signal received by the receiving means.

Here, there may be constructed a constitution including reception interval setting means for setting the reception interval of the receiving means, wherein the receiving means receives the bionic information signal transmitted by the transmission interval in synchronism with the reception interval set by the reception interval setting means, and the noticing means carries out the notice indicating the bionic information of the measured person based on the received bionic information signal.

Further, there may be constructed a constitution in which the reception interval setting means is constituted by including at least one of operating means capable of setting the reception interval, receiver power source state detecting means for detecting a state of a power source for the receiver and setting the reception interval in accordance with the state of the power source, and mode detecting means for detecting an operation mode of counting means provided on a side of the receiver and setting the reception interval in accordance with the operation mode of the counting means.

Further, there may be constructed a constitution in which in a case of including at least the receiver power source state detecting means as the reception interval setting means, when the receiver power source state detecting means detects that the power source for the receiver is reduced to be equal to or smaller than a predetermined voltage and the reception interval is set, the reception interval is set the most predominantly by the receiver power source state detecting means.

According to the bionic information measuring system according to the invention, the receiver can be constituted by low power consumption formation.

Further, according to the invention, the transmitter for measuring bionic information and the receiver for measuring bionic information suitable for constructing the bionic information measuring system can be provided.

### BRIEF DESCRIPTION OF THE DRAWINGS

FIG. 1 is a block diagram of a heartbeat meter system according to a first embodiment of the invention.

FIG. 2 is a state table used in the first embodiment of the invention.

FIG. 3 is a diagram showing a format of a heart rate information signal used in the first embodiment of the invention.

FIG. 4 is a diagram showing a reception timing of the first embodiment of the invention.

FIG. 5 is a flowchart showing processing of the first embodiment of the invention.

FIG. 6 is a flowchart showing processing of the first embodiment of the invention.

FIG. 7 is a flowchart showing processing of the first embodiment of the invention.

FIG. 8 is a block diagram of a heartbeat meter system according to a second embodiment of the invention.

FIG. 9 is a transmitting timing table used in the second embodiment of the invention.

FIG. 10 is a diagram showing a format of a first heart rate information signal used in the second embodiment of the invention.

FIG. 11 is a diagram showing a format of a second heart rate information signal used in the second embodiment of the invention.

FIG. 12 is a timing chart in the second embodiment of the invention.

FIG. 13 is a state table used in the second embodiment of the invention.

FIG. 14 is a flowchart showing processing of the second embodiment of the invention.

FIG. 15 is a flowchart showing processing of the second embodiment of the invention.

FIG. 16 is a flowchart showing processing of the second embodiment of the invention.

FIG. 17 is a flowchart showing processing of the second embodiment of the invention.

### DESCRIPTION OF THE PREFERRED EMBODIMENTS

An explanation will be given of a bionic information measuring system, a bionic information measuring transmitter and a bionic information measuring receiver according to an embodiment of the invention as follows.

Further, according to the embodiment, as the bionic information measuring system, there is pointed out an example of a portable heartbeat measuring system for informing a heart rate by mounting a heartbeat meter main body (bionic information measuring transmitter) for transmitting in wireless a heartbeat information signal by detecting heartbeat to the chest of a user by a chest belt in a press contact state, using a wristwatch type receiver (bionic information measuring receiver), and receiving the heartbeat information signal from the heartbeat main body by the wristwatch type receiver.

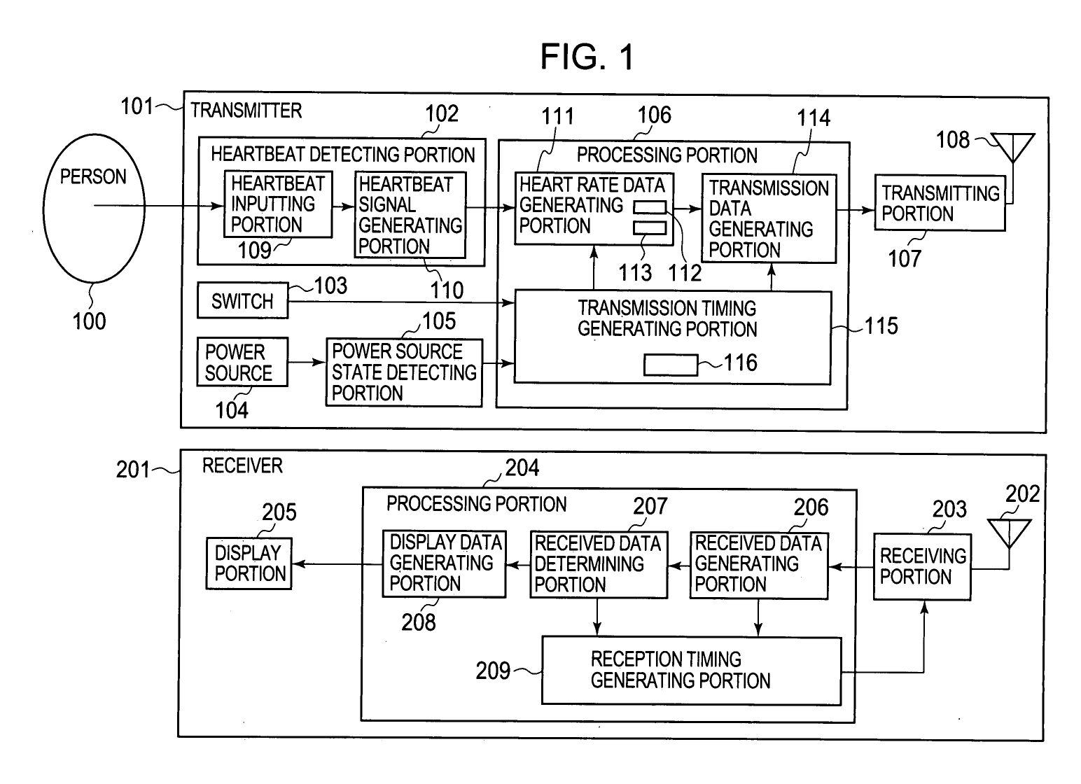

FIG. 1 is a block diagram of a heartbeat measuring system according to a first embodiment of the invention.

In FIG. 1, the heartbeat measuring system includes a transmitter 101 as a bionic information measuring transmitter for detecting the heartbeat of a measured person 100 constituting a bionic signal and transmitting a heartbeat information signal (bionic information signal) including heartbeat information (bionic information) indicating information of the heartbeat and transmission interval information of transmitting the heartbeat information, and a receiver 201 as a bionic information measuring receiver for receiving a heartbeat information signal from the transmitter 101 by a timing in synchronism with a transmission interval included in the heartbeat information signal.

The transmitter 101 is integrally mounted to a chest belt (not illustrated) and is used by being mounted to the chest of the measured person 100. The receiver 201 is provided with an outer shape of a wristwatch type and is used by being mounted to the arm of the measured person 100. The transmitter 101 and the receiver 201 are constituted to be able to communicate with each other, and one way transmission of the heartbeat information signal is carried out from the transmitter 101 to the receiver 201 by wireless (for example, electromagnetic induction).

The transmitter 101 detects heartbeat of the measured person 100 and transmits the heartbeat information signal constituted by adding the transmission interval information to the heartbeat information in correspondence with the heart rate per unit time to the receiver 201 by the transmission interval. The receiver 201 is constituted to receive the heartbeat signal information by a timing in synchronism with the transmission interval and carry out a notice of a display of the heart rate constituting the heartbeat information or the like.

The transmitter 101 includes a heartbeat detecting portion 102 for outputting a pulse signal of a rectangular wave in correspondence with the heartbeat signal at each time of detecting the heartbeat signal, a switch 103 for setting the transmission interval of the heartbeat information, a power source 104 for supplying a drive power to respective circuit elements of the transmitter 101, a power source detecting portion 105 for detecting a situation (for example, voltage) of the power source 104 and noticing the situation of the power source 104, a processing portion 106 for generating the heartbeat information signal generating the heartbeat information constituting the heart rate per unit time by counting the pulse signal from the heartbeat detecting portion 102 and adding the transmission interval information indicating the transmission interval of transmitting the heartbeat information and then, outputting the heartbeat information signal by the transmission terminal, a transmitting portion 107 for modulating the heartbeat information signal from the processing portion 106 to transmit to outside, and an antenna 108 for transmitting the signal from the transmitting portion 107 to outside by wireless.

Either of a primary battery or a secondary battery can be used for the power source 104.

The heartbeat detecting portion 102 includes a heartbeat inputting portion 109 which is a sensor for detecting and outputting the heartbeat signal of the measured person 100, and a heartbeat signal generating portion 110 for shaping the heartbeat signal from the heartbeat inputting portion 109 into a detected pulse signal of a rectangular wave to output.

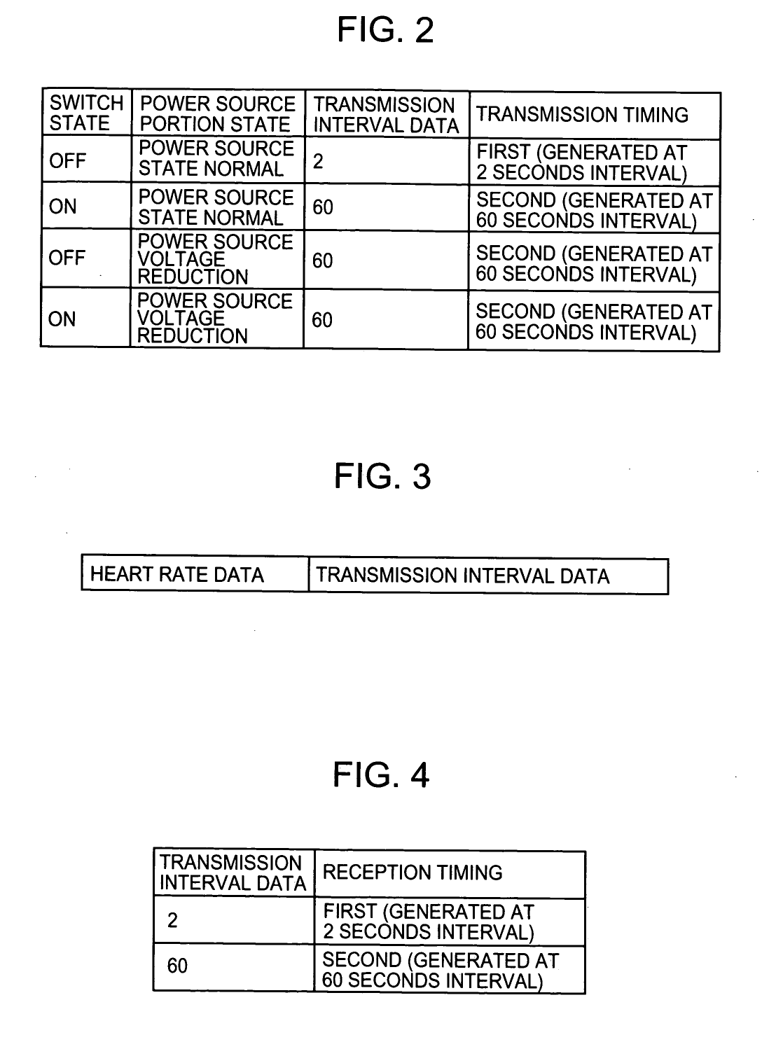

The processing portion 106 includes a heart rate data generating portion 111 for calculating a heart rate per unit time by counting the detected pulse signal from the heartbeat detecting portion 102 by a counter 112, storing the heart rate to a memory 113 as the heartbeat information and outputting the heart rate as the heartbeat information of a serial type, a transmitting data generating portion 114 for generating the heartbeat information signal of FIG. 3 by adding the transmission interval information to the heartbeat information from the heart rate data generating portion 111, and a transmission timing generating portion 115 for storing a state table of FIG. 2 to a memory 116 and controlling to supply a transmission timing control signal to the heart rate data generating portion 111 and the transmission data generating portion 114 to output the heartbeat information signal by the set transmission timing.

In FIG. 2, the state table stored to the memory 116 includes, as items, a state of the switch 103 (ON or OFF), a state of the power source 104 (power state normal indicating that the power source state is normal, power source voltage reduction indicating that a power source voltage is equal to or smaller than a predetermined value), a data indicating the transmission interval (“2” indicating a first timing, “60” indicating a second timing), and the transmission timing (first timing (2 seconds interval) or a second timing (60 seconds interval)). The transmission interval is constituted to determine the power source state the most predominantly, in a case of a state in which the power source voltage of the power source 104 is equal to or lower than a predetermined voltage, in either of cases of ON and OFF of the state of the switch 103, the transmission timing is constituted to be set to the second timing having the longer transmission interval to restrain power consumption.

Although a detailed operation will be described later, the transmitter 101 calculates the heart rate per a predetermined time period by detecting heartbeat of the measured person 100, and transmits in wireless the bionic information signal of FIG. 3 including the heartbeat information and the transmission interval information by the transmission interval in accordance with the state of setting the switch 103 and the state of the power source 104 in reference to the state table of FIG. 2 stored to the memory 116.

Further, the heartbeat detecting portion 102 constitutes sensor means, the processing portion 106, the transmission portion 107 and the antenna 108 constitute transmitting means, and the switch 103 and the power source state detecting portion 105 constitute transmission interval setting means. Further, the switch 103 constitutes operating means, and the power source state detecting portion 105 constitutes transmitter power source state detecting means.

On the other hand, the receiver 201 includes an antenna 202, a receiving portion 203 for demodulating and outputting the heartbeat information signal received by the antenna 202, a processing portion 204 for extracting to output the heartbeat information included in the heartbeat information signal received by the receiving portion 203 and controlling a reception timing of the receiving portion 203 in synchronism with the transmission interval information included in the heartbeat information signal, and a display portion 205 for displaying to display the heartbeat information from the processing portion 204.

The processing portion 204 includes a received data generating portion 206 for extracting the heartbeat information and the transmission interval information from the heartbeat information signal from the receiving portion 203 to convert into a serial data signal, a received data determining portion 207 for outputting the heartbeat information from the received data generating portion 206 to a display data generating portion 208 and determining the transmission interval information and outputting a reception timing setting signal for setting a reception timing to a reception timing generating portion 209, the display data generating portion 208 for converting the heartbeat information from the received data determining portion 207 to a displayable signal style to output to the display portion 205, and the reception timing generating portion 209 for controlling a reception interval of the receiving portion 203 by the reception timing as shown by FIG. 4 in accordance with the reception timing setting signal from the received data determining portion 207.

The receiver 201 receives the heartbeat information signal from the transmitter 101 by the reception timing in synchronism with the transmission interval information included in the heartbeat information signal to display by the display portion 205 to indicate the heartbeat information included in the heartbeat information signal although a detailed operation thereof will be described later.

Further, the antenna 202 and the receiving portion 203 constitute receiving means, the processing portion 204 constitutes controlling means, and the display portion 205 constitutes noticing means.

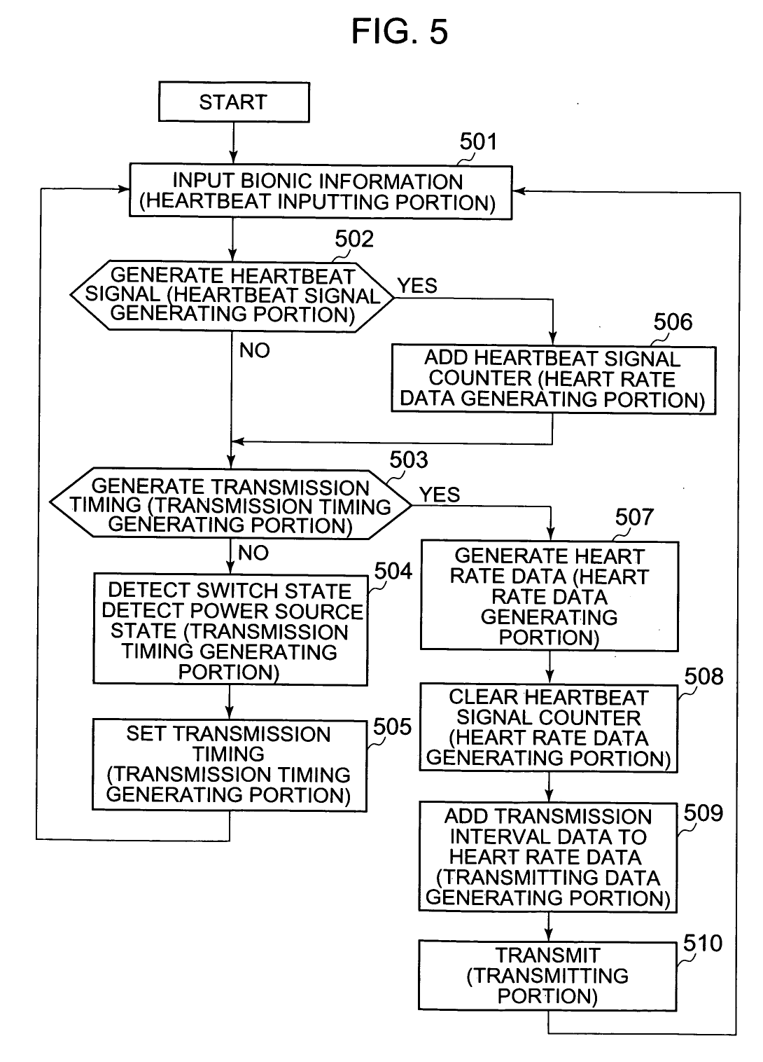

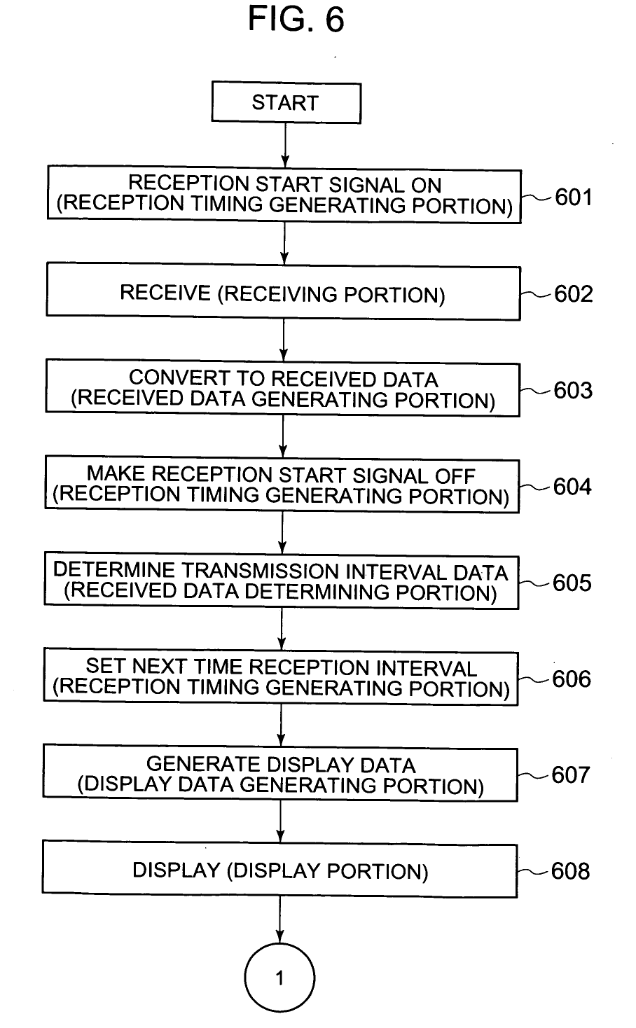

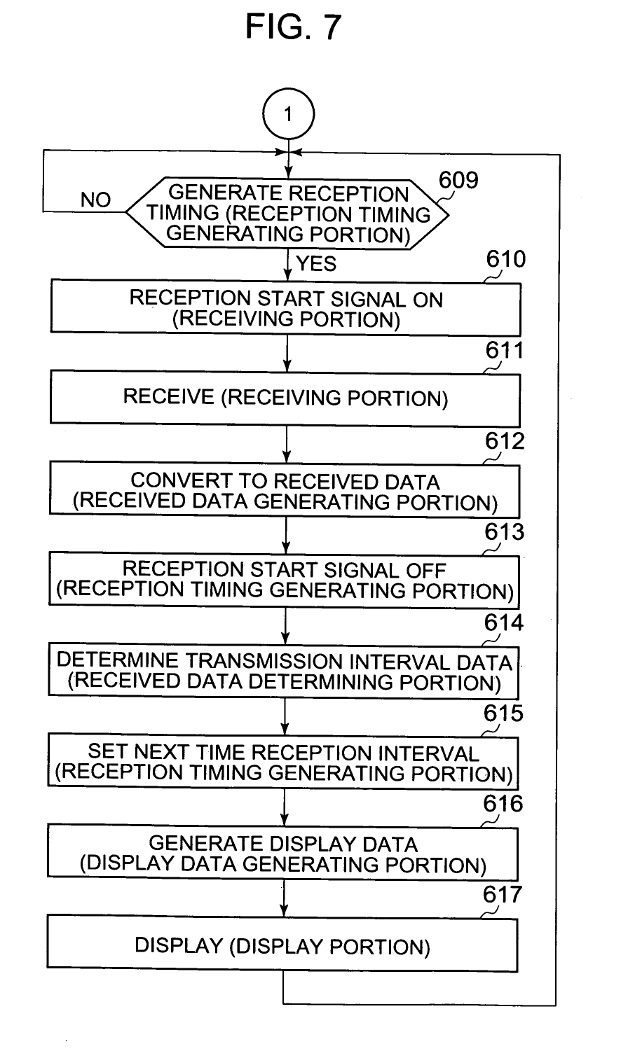

FIG. 5 is a flowchart showing processing of the transmitter 101. Further, FIG. 6 and FIG. 7 are flowcharts showing processing of the receiver 201.

An explanation will be given of operation of the first embodiment of the invention in reference to FIG. 1 through FIG. 7 as follows.

The heartbeat inputting portion 109 detects and outputs a heartbeat signal constituting a bionic signal of the measured person 100 (step S501 of FIG. 5).

In a case which the heartbeat signal generating portion 110 determines that the heartbeat signal is not inputted from the heartbeat inputting portion 109 (step S502), when the transmission timing generating portion 115 determines that the transmission timing is not generated (step S503), in reference to the state table of FIG. 2 stored to the memory 116, the transmission timing is set based on a state of detecting a state of the switch 103 and a state of detecting the power source 104 by the power source state detecting portion 105 to return to the processing step S501 (step S504, S505).

When it is determined that the heartbeat signal is inputted from the heartbeat inputting portion 109 at processing step S502, the heartbeat signal generating portion 110 adds 1 to a counted value of the heartbeat signal counter 112 (step S506) and proceeds to processing step S503 (step S506).

As described above, during the transmission interval until the transmission timing arrives, operation of counting the heartbeat signal and operation of setting the transmission timing are repeated.

When it is determined that the transmission timing arrives at processing step S503, the transmission timing generating portion 115 outputs the transmission timing signal to the heart rate data generating portion 111.

In response to the transmission timing signal, the heart rate data generating portion 111 generates the heartbeat information constituting the heart rate per a predetermined time period based on the counted value of the counter 112, stores the heartbeat information to the memory 113 and outputs it to the transmission data generating portion 114 (step S507), thereafter, clears the heartbeat signal counter 112 (step S508).

Simultaneously therewith, in response to the transmission timing signal, the transmission data generating portion 114 generates the heartbeat information signal of FIG. 3 by adding the transmission interval information set by the transmission timing generating portion 115 to the heartbeat signal inputted from the heart rate data generating portion 111 and outputs the heartbeat information signal to the transmitting portion 107 (step S509).

The transmitting portion 107 transmits in wireless the heartbeat information signal to outside by way of the antenna 108 (step S510).

On the other hand, on the side of the receiver 201, the reception timing generating portion 209 makes a reception start signal for controlling a receiving operation of the receiving portion 203 ON to make the receiving portion 203 carry out the receiving operation (step S601 of FIG. 6). At this occasion, since the transmission timing from the transmitter 101 is unknown until receiving a first signal from the transmitter 101, during a time period until receiving the first signal from the transmitter 101, the reception timing generating portion 209 controls the receiving portion 203 to continue the receiving operation.

When the heartbeat information signal is received by way of the antenna 202, the receiving portion 203 demodulates the heartbeat information signal to output to the received data generating portion 206 (step S602). The received data generating portion 206 converts the heartbeat signal and the transmission interval information (refer to FIG. 4) included in the heartbeat information signal from the receiving portion 203 into a serial data signal to output to the received data determining portion 207 and the reception timing generating portion 209 (step S603).

In response to the signal from the receiving portion 203, the reception timing generating portion 209 stops supplying power source to the receiving portion 203 by making the reception start signal OFF to stop a receiving operation of the receiving portion 203 (step S604). Thereby, power consumption of the receiving portion 203 is dispensed with.

The received data determining portion 207 determines the transmission interval from the transmission interval information (step S605), and the reception timing generating portion 209 sets a reception interval at a next time to the reception interval in synchronism with the transmission interval determined by the received data determining portion 207 (step S606).

For example, as shown by FIG. 4, when the transmission interval information is “2”, the reception timing is set to a first timing (reception timing is 2 seconds interval), when the reception interval information is “60”, the reception timing is set to a second timing (reception timing is 60 seconds interval).

The reception timing generating portion 209 supplies power source to the receiving portion 203 for a predetermined time period intermittently by the set reception timing, makes the receiving portion 203 carry out the receiving operation intermittently by the reception interval and receive the heartbeat information signal from the transmitter 101. Thereby, the receiving portion 203 receives the heartbeat information signal by carrying out the receiving operation intermittently by a timing in synchronism with the transmission timing on the side of the transmitter 101.

When the heartbeat information is inputted from the received data determining portion 207, the display data generating portion 208 generates a display signal for displaying the heart rate in correspondence with the heartbeat information (step S607), and the display portion 205 displays the heart rate in correspondence with the display signal (step S608).

Next, when it is determined that the reception timing arrives (step S609 of FIG. 7), the reception timing generating portion 209 makes the receiving portion 203 carry but a receiving operation by making the reception start signal for controlling the receiving operation of the receiving portion 2030N (step S610).

When the heartbeat information signal is received by way of the antenna 202, the receiving portion 203 demodulates the heartbeat information signal to output to the received data generating portion 206 (step S611). The received data generating portion 206 converts the heartbeat information and the transmission interval information (refer to FIG. 4) included in the heartbeat information signal from the receiving portion 203 into a serial data signal to output to the received data generating portion 206 and the reception timing generating portion 209 (step S612).

In response to the signal from the receiving portion 203, the reception timing generating portion 209 stops supplying power source to the receiving portion 203 by making the reception start signal OFF to stop the receiving operation of the receiving portion 203 (step S613).

The received data determining portion 207 determines the transmission interval from the transmission interval information (step S614), and the reception timing generating portion 209 sets a reception interval at a next time to a reception interval coinciding with the transmission interval determined by the input data determining portion 207 (step S615).

The reception timing generating portion 209 supplies power source to the receiving portion 203 for a predetermined time period by the set reception timing, makes the receiving portion 203 carry out the receiving operation and receives the heartbeat information signal from the transmitter 101. Thereby, the receiving portion 203 carries out the receiving operation at timing in synchronism with the transmission timing on the side of the transmitter 101 to receive the heartbeat information signal.

When the heartbeat information is inputted from the received data determining portion 207, the display data generating portion 208 generates a display signal for displaying the heart rate (step S616), the display portion 205 displays the heart rate in correspondence with the display signal (step S617), thereafter, returns to processing step S609 to repeat the processing. Thereby, the receiving portion 203 is operated intermittently in synchronism with the transmission timing of the heartbeat information signal of the transmitter 101, the heartbeat information signal is received and the heart rate is updated at a predetermined interval to display by the display portion 205.

As described above, according to the heartbeat measuring system of the first embodiment, on the side of the transmitter 101, the heartbeat signal (bionic signal) of the measured person 100 is detected to detect the heart rate per the predetermined time period, the state table of FIG. 2 stored to the memory 116 is referred, the heartbeat information signal (bionic information signal) of FIG. 3 including the heartbeat information (bionic information) and the transmission interval information is transmitted in wireless by the transmission interval in accordance with the state of setting the switch 103 and the state of the power source 104, on the other hand, on the side of the receiver 201, the heartbeat information signal (bionic information signal) from the transmitter 101 is received by the reception timing in synchronism with the transmission interval information included in the heartbeat information signal (bionic information signal) and is displayed by the display portion 205 to indicate the heartbeat information (bionic information) included in the heartbeat information signal (bionic information signal).

Therefore, when the heartbeat information signal (bionic information signal) from the transmitter 101 is received by the receiver 201, the receiving operation of the receiver 201 can be carried out intermittently, and therefore, the side of the receiver 201 can be constituted by low power consumption formation.

Therefore, the heartbeat measuring system capable of constituting the side of the receiver 201 by low power consumption formation can be provided. Further, the transmitter 101 and the receiver 201 suitable for constructing the heartbeat measuring system can be provided.

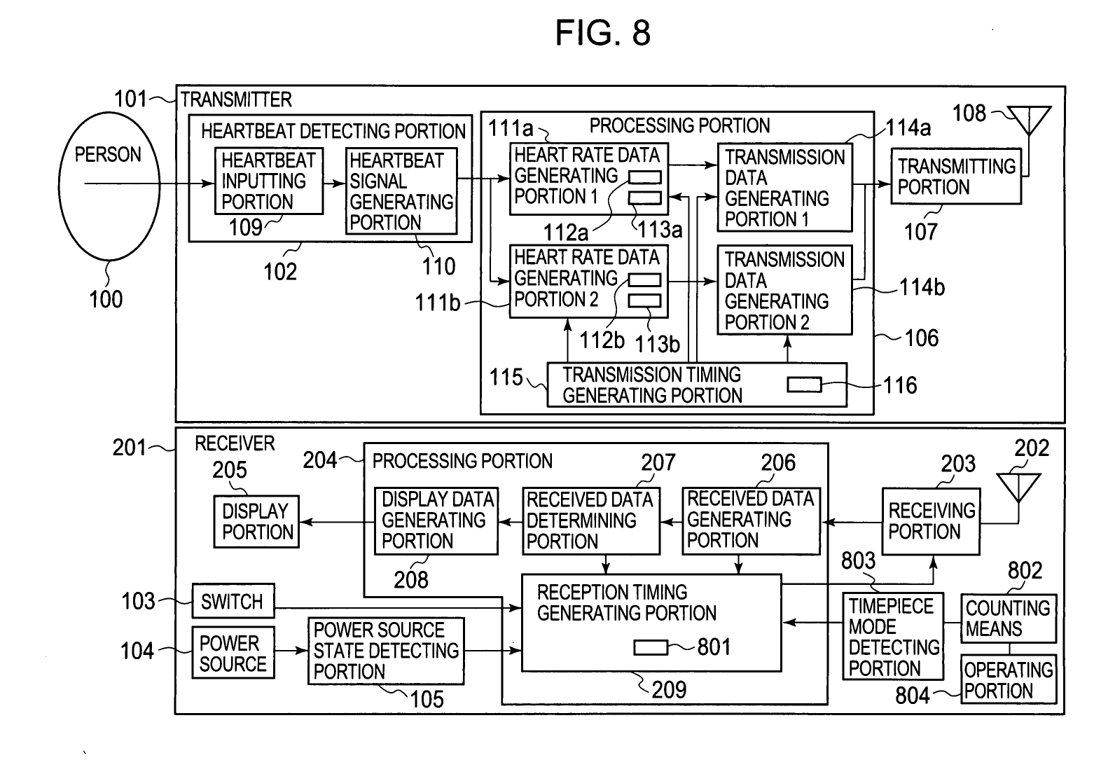

FIG. 8 is a block diagram of a bionic information measuring system according to a second embodiment of the invention showing an example of a heartbeat measuring system similar to the first embodiment, and portions having portions the same as those of FIG. 1 are attached with the same notations (when there are a plurality of portions having the same functions, the portions are differentiated from each other by attaching notations a and b.

Although according to the first embodiment, a timing by which the receiver receives the bionic information signal is constituted to be set by the side of the transmitter, according to the second embodiment, a timing of receiving the bionic information signal is constituted to set by the side of the receiver. An explanation will mainly be given of portions which differ from those of the first embodiment.

In FIG. 8, the heartbeat measuring system includes the transmitter 101 as a bionic information measuring transmitter for detecting the heartbeat vibration of the measured person 100 which is the bionic information and transmitting the heartbeat information signal (bionic information signal) including the heartbeat information (bionic information) indicating the information of the heartbeat signal and the transmission interval information of transmitting the heartbeat information by a plurality of transmission intervals which differ from each other, and the receiver 201 as a bionic information measuring receiver for determining by which transmission interval transmitted heartbeat information signal is received and receiving the heartbeat information signal by a timing in synchronism with the heartbeat information signal transmitted by the predetermined interval.

The processing portion 106 of the transmitter 101 includes a first heart rate data generating portion 111a for calculating a heart rate per unit time by a first heartbeat signal counter 112a from the detected pulse signal from the heartbeat detecting portion 102, storing the heart rate to a first memory 113a as the heart beat information and outputting the heartbeat rate as heartbeat information in a serial style, and a first transmission data generating portion 114a for generating a first heartbeat information signal of FIG. 10 by adding transmission interval information to the heartbeat information from the first heart rate data generating portion 111a.

Further, the processing portion 106 of the transmitter 101 includes a second heart rate data generating portion 111b for calculating a heart rate per unit time by a second heart rate signal counter 112b from the detected pulse signal from the heartbeat detecting portion 102, storing the heart rate to a second memory 113b as heartbeat information and outputting the heartbeat rate as heartbeat information in a serial style, and a second transmitting data generating portion 114b for generating a second heartbeat information signal of FIG. 11 by adding transmission interval information to the heartbeat information from the second heart rate data generating portion 111b.

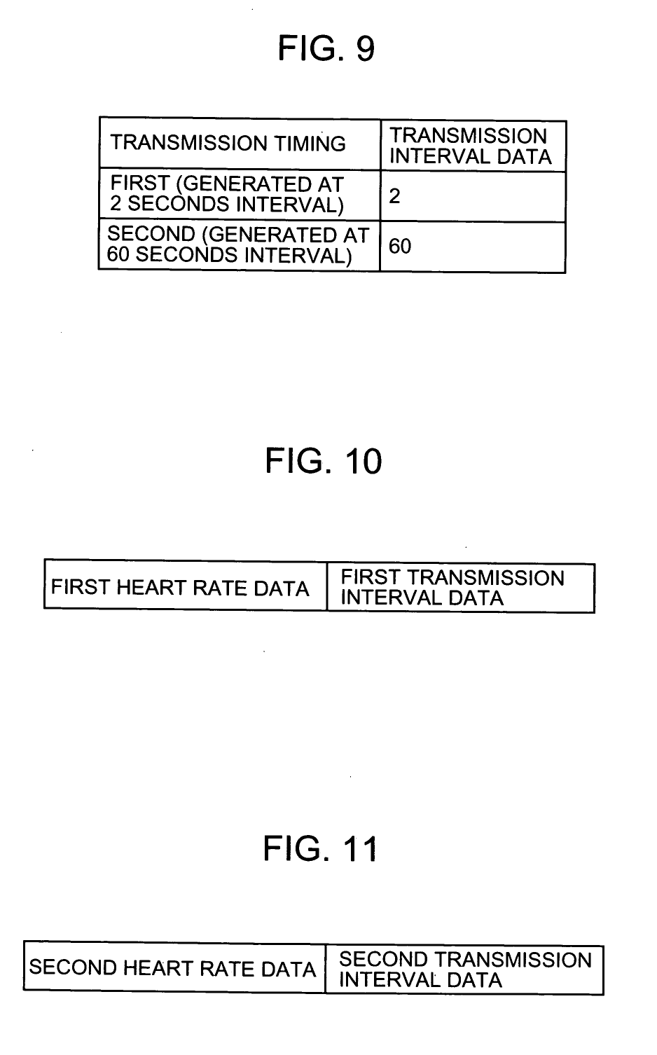

Further, the processing portion 106 of the transmitter 101 includes the transmission timing generating portion 115 for storing a transmission timing table of FIG. 9 to the memory 116, controlling to transmit the heartbeat information signal by controlling the first heart rate data generating portion. 111a and the first transmission data generating portion 114a by a first transmission timing (2 seconds interval according to the embodiment), controlling to transmit the heartbeat information signal by controlling the second heart rate data generating portion 111b and the second transmission data generating portion 114b by a second transmission timing (60 seconds interval according to the embodiment).

FIG. 9 includes data indicating a transmission timing (2 seconds interval for the first timing, 60 seconds interval for the second timing) at the table stored to the memory 116.

Although a detailed operation will be descried later, on the side of the transmitter 101, a heart rate per unit time is calculated by detecting the heartbeat of the measured person 100, the transmission timing generating portion 115 refers to the state table of FIG. 2 stored to the memory 116, and the transmission timing generating portion 115 controls the transmission timing such that a first heart rate information signal of FIG. 10 generated by the heart rate data generating portion 111a and the transmission data generating portion 114a is transmitted by a first transmission interval (according to the embodiment, 2 seconds interval) and a second heart rate information signal of FIG. 11 generated by the heart rate generating portion 111b and the transmission data generating portion 114b is transmitted by a second transmission interval (according to the embodiment, 60 seconds interval).

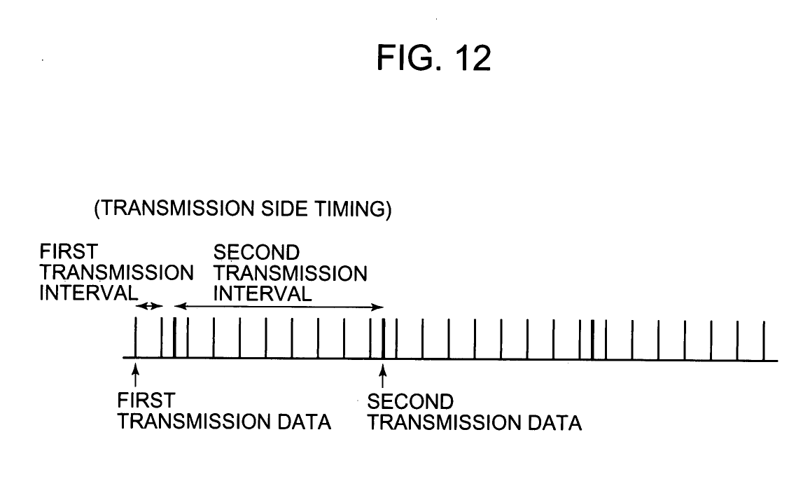

FIG. 12 is a timing chart when the transmitter 101 transmits the bionic information signal.

In FIG. 12, the transmitter 101 transmits the first bionic information signal by the first transmission interval (for example, 2 seconds interval) and transmits the second bionic information signal by the second transmission interval (for example, 60 seconds interval). A deviation between the first transmission interval and the second transmission interval is set to a value shorter than the first transmission interval.

On the other hand, the receiver 201 includes the switch 103, the power source 104 for supplying drive power to respective constituent elements of the receiver 201, the power source state detecting portion 105 for detecting a power source state of a power source voltage or the like of the power source 104, counting means 802 for carrying out timepiece operation, a mode detecting portion 803 for detecting a mode of setting the counting means (for example, a stopwatch mode constituting, for example, a kind of an application state), and an operating portion 804 for setting the mode of the counting means.

Either of a primary battery or a secondary battery can be used for the power source 104.

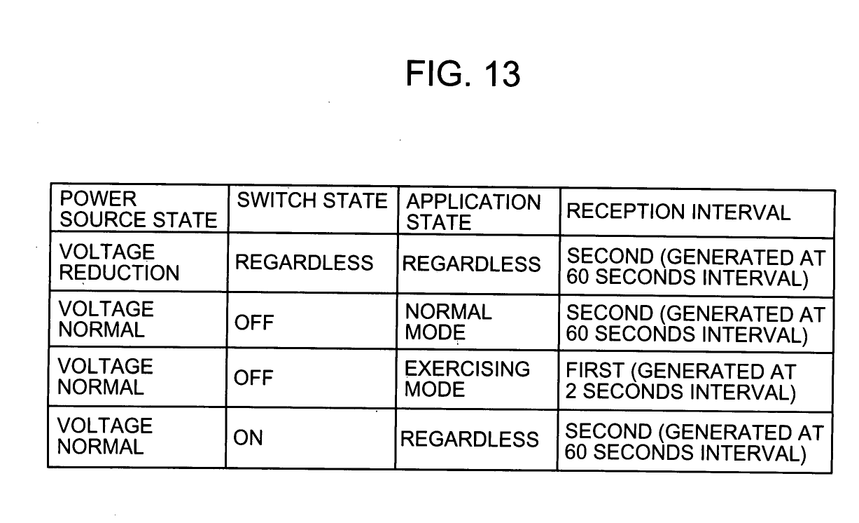

Further, the reception timing generating portion 209 includes a memory 801 for storing a state table of FIG. 13. The state table of FIG. 13 is provided with, as items, the power source state of the power source 104 (a state indicating whether the voltage is normal or is equal to or smaller than a predetermined voltage), a state of the switch 103 (ON state, OFF state, regardless), an application state indicating the mode of the counting means 802 (regardless, normal mode constituting a mode of counting time, exercising mode of carrying out stopwatch operation), reception interval (the first reception interval is 2 seconds interval, the second reception interval is 60 seconds) in correspondence with each other.

According to the second embodiment, the reception interval is set in consideration of the state of the power source 104 the most predominantly. That is, as shown by the state table of FIG. 13, in any state of other parameter for setting the reception interval of the switch 103 or the like, when the voltage of the power source 104 is reduced to be equal to or smaller than the predetermined value, the reception interval is constituted to set a maximum reception interval (second receiving interval 60 seconds). Thereby, even when a remaining amount of the power source 104 is reduced, the power source 104 is made to be able to be used longer by restraining power consumption.

Although a detailed operation will be described later, the receiver 201 selects the receiving interval based on the state of the switch 103 or the power source 104 or the operation mode of the counting means 802, receives the heartbeat information signal transmitted by either of the transmission intervals of the heartbeat information signals transmitted from the transmitter 101 by the plurality of transmission intervals, and displays the heartbeat information included in the heartbeat information signal by the display portion 205.

Further, the switch 103, the power source state detecting portion 105, the timepiece mode detecting portion 803 and the operating portion 804 constitute receiving interval setting means. Further, the switch 103 constitutes operating means, the power source state detecting portion 105 constitutes receiver power source state detecting means, the timepiece mode detecting means 803 constitutes mode detecting means.

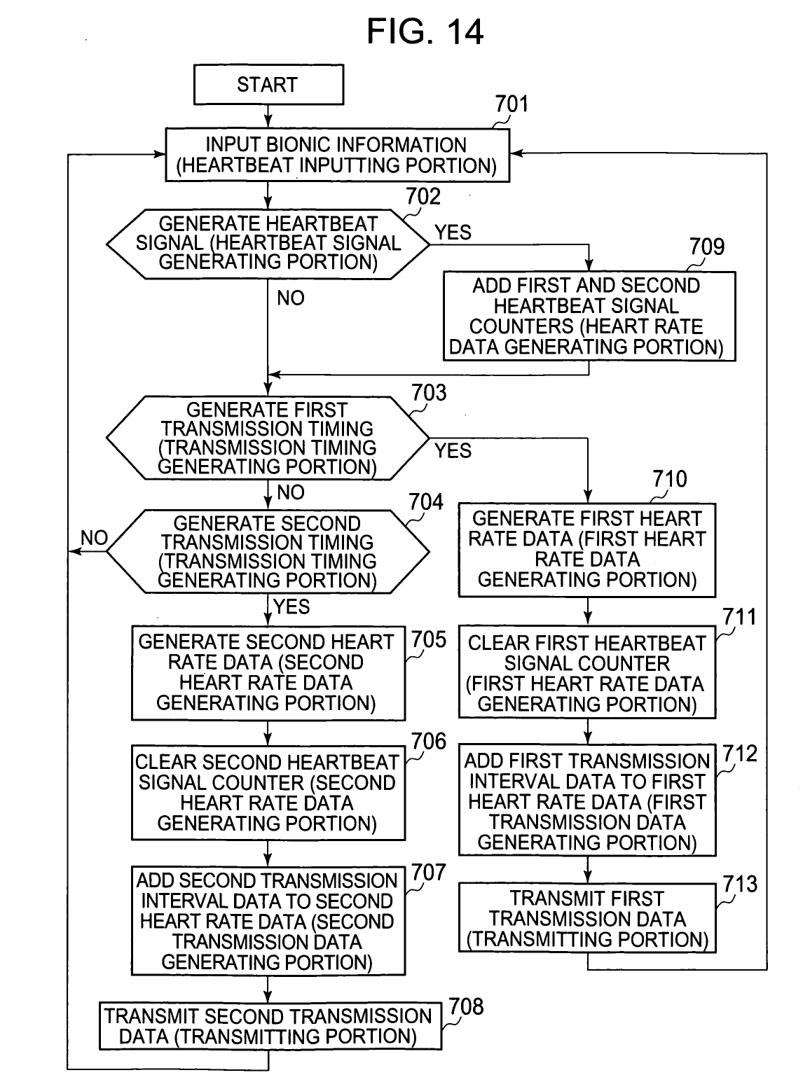

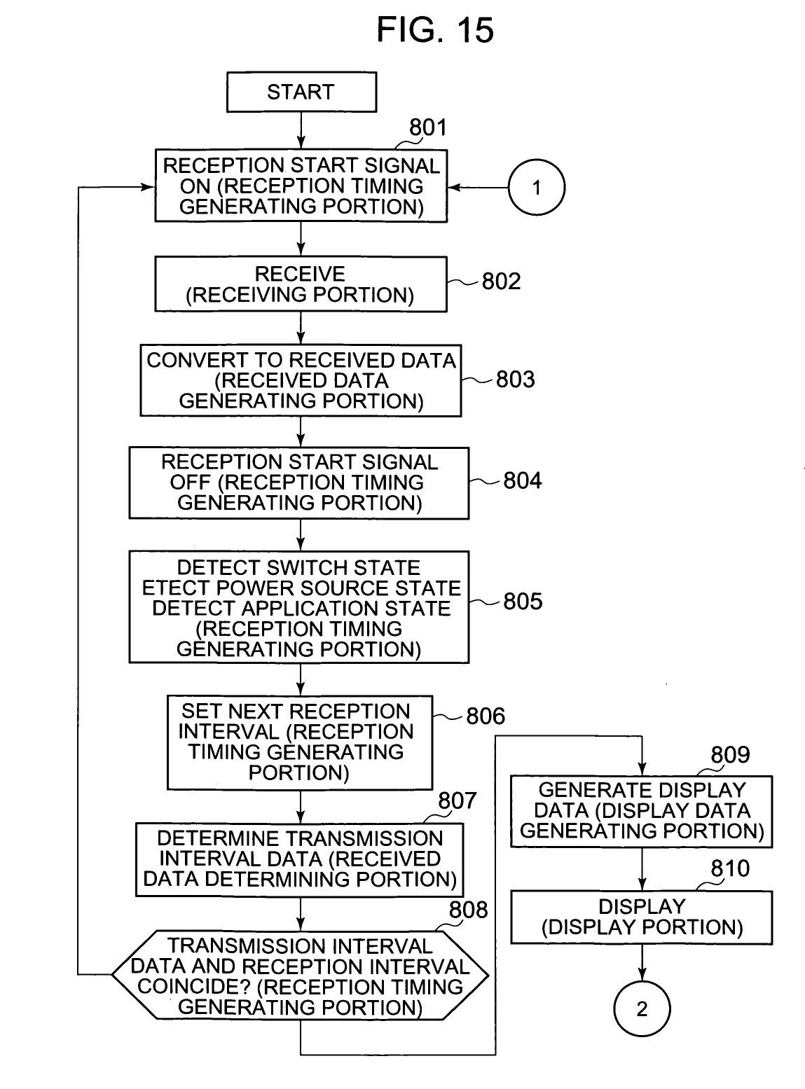

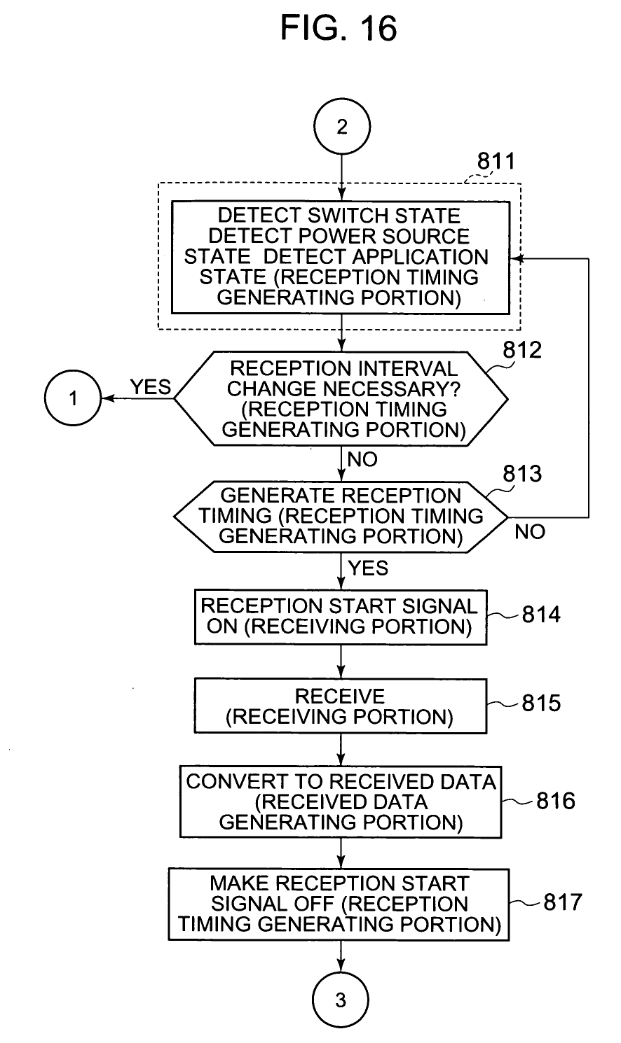

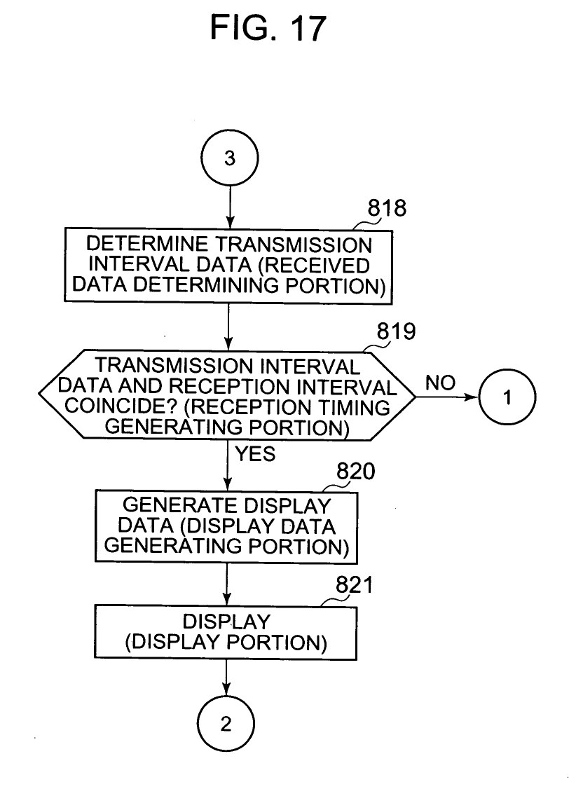

FIG. 14 is a flowchart showing processing of the transmitter 101, and FIGS. 15 to 17 are flowcharts showing processing of the receiver 201.

Operation of the second embodiment of the invention will be explained in reference to FIG. 8 through FIG. 17 as follows.

The heartbeat inputting portion 109 detects to output the heartbeat signal of the measured person 100 (step S701).

In a case in which the heartbeat signal generating portion 110 determines that the heartbeat signal is not inputted from the heartbeat inputting portion 109 (step S702), when it is determined that the first transmission timing does not arrive (step S703), the transmission timing generating portion 115 determines whether the second transmission timing arrives (step S704).

When it is determined that the second transmission timing does not arrive at processing step S704, the transmission timing generating portion 115 returns to processing step S701, when it is determined that the second transmission timing arrives, the second heartbeat data generating portion 111b controls to generate the second heartbeat information based on the counted value of the second heartbeat signal counter 112b and stores the heart rate constituting the counted value to the memory 113b (step S705), thereafter, clears the counted value of the second heartbeat signal counter 112b (step S706) and controls to output the second heartbeat information signal shown in FIG. 11 by adding the second transmission interval information to the second heart rate information (step S707).

The transmitting portion 107 transmits in wireless the second heartbeat information signal (second transmitting data) by way of the antenna 108, thereafter returns to processing step S701 (step S708).

When it is determined that the heartbeat signal is inputted from the heartbeat inputting portion 109 at processing step S702, the heartbeat signal generating portion 110 adds 1 respectively to counted values of the first and the second heartbeat signal counters 112a and 112b to proceed to processing step S703 (step S709).

When it is determined that the first transmission timing arrives at processing step S703, the transmission timing generating portion 115 outputs the transmission timing signal to the first heart rate data generating portion 111a.

In response to the transmission timing signal, the first heart rate data generating portion 111a generates the heartbeat information constituting the heart rate per predetermined time period based on the counted value of the counter 112a, stores the heartbeat information to the memory 113a and outputs the heartbeat information to the first transmission data generating portion 114a (step S710), thereafter, clears the first heartbeat signal counter 112a (step S711).

In response to the transmission timing signal, the transmission data generating portion 114 generates the heart rate information signal of FIG. 10 by adding the transmission interval information set by the transmission timing generating portion 115 to the heartbeat information inputted from the first heart rate data generating portion 111 and outputs the heart rate information signal to the transmitting portion 107 (step S712).

The transmitting portion 107 transmits in wireless the first heart rate information signal (first transmission data) to the receiver 201, thereafter, returns to processing step S701 (step S713). The transmitter 101 transmits the heart rate information signals by the plurality of transmission intervals by repeating the above-described processing.

As described above, the transmitter 101 transmits the first and the second heartbeat information signals by the transmission intervals shown in FIG. 12.

On the other hand, on the side of the receiver 201, the reception timing generating portion 209 makes a reception start signal for controlling a receiving operation of the receiving portion 203 ON and makes the receiving portion 203 carry out the receiving operation (step S801 of FIG. 15). At this occasion, the transmission timing from the transmitter 101 is unknown until a first signal is received from the transmitter 101, the reception timing generating portion 209 controls the receiving portion 203 to continue the receiving operation until the first signal is received from the transmitter 101.

When the heartbeat information signal is received by way of the antenna 202, the receiving portion 203 demodulates the heartbeat information signal to output to the received data generating portion 206 (step S802). The receiving data generating portion 206 converts the heartbeat information and the transmission interval information (refer to FIG. 10, FIG. 11) included in the heartbeat information signal from the receiving portion 203 into a serial data signal to output to the received data determining portion 207 and the reception timing generating portion 209 (step S803).

In response to the signal from the receiving portion 203, the reception timing generating portion 209 stops supplying power source to the receiving portion 203 by making the reception start signal OFF to stop the receiving operation of the receiving portion 203 (step S804).

Next, the reception timing generating portion 209 detects the state of the power source 104 by detection of the power source state detecting portion 105, the state of the switch 103 and an application state (according to the embodiment, the mode of the counting means 802 detected by the timepiece mode detecting means 803) (step S805), and sets a reception interval at a next time toga reception interval in correspondence with the respective states in reference to the state table of FIG. 13 stored to the memory 801 (step S806).

The received data determining portion 207 determines the transmission interval from the received transmission interval information (step S807), the reception timing generating portion 209 determines whether the transmission interval information and the set reception interval coincide with each other (step S808), in a case of incoincidence, returns to processing step S801 to carry out the processing again, in a case of coincidence, determines that the transmitting operation on the side of the transmitter 101 and the receiving operation on the side of the receiver 201 are synchronized, the display data generating portion 208 generates the display signal for displaying the heartbeat information when the heartbeat information is inputted from the received data determining portion 207 (step S809), and the display portion 205 displays the heart rate in correspondence with the display signal (step S810).

Next, the reception timing generating portion 209 detects a state of the switch 103, a state of the power source 104 by detection of the power source state detecting portion 105, and an application state (step S811).

The reception timing generating portion 209 determines the reception interval in correspondence with results of the respective states in reference to the state table of FIG. 13 stored to the memory 801 and determines whether it is necessary to change the reception interval (step S812).

The reception timing generating portion 209 returns to processing step S801 when it is determined that it is necessary to change the receiving interval at processing step S811, determines whether the reception timing arrives when it is determined that it is not necessary to change the reception interval (step S813), returns to step S811 to detect the states when the reception timing does not arrive, when it is determined that the reception timing arrives (reception timing is generated), makes the reception start signal for controlling the receiving operation of the receiving portion 203 ON and makes the receiving portion 203 carry out the receiving operation by supplying power source (step S814).

When the heartbeat information signal is received by way of the antenna 202, the receiving portion 203 demodulates the heartbeat information signal to output to the received data generating portion 206 (step S815).

The received data generating portion 206 converts the heartbeat information and the transmission interval information included in the heartbeat information signal from the receiving portion 203 into a serial data signal to output to the received data determining portion 207 and the reception timing generating portion 209 (step S816).

In response to the signal from the receiving portion 203, the reception timing generating portion 209 stops supplying power source to the receiving portion 203 by making the reception start signal OFF to stop the receiving operation of the receiving portion 203 (step S817).

The received data determining portion 207 determines the transmission interval from the transmission interval information (step S818), the reception timing generating portion 209 determines whether the received transmission interval information and the reception interval coincide with each other (step S819), in a case of incoincidence, proceeds to processing step S801, in a case of coincidence, the display data generating portion 208 generates the display signal for displaying the heartbeat information when the heartbeat information is inputted from the received data determining portion 207 (step S820), and the display portion 205 displays the heart rate in correspondence with the display signal (step S821), thereafter, returns to processing step S811 to repeat the processing.

As descried above, according to the heartbeat measuring system of the second embodiment, the heartbeat measuring system is constituted such that on the side of the transmitter 101, the heart rate (bionic signal number) per predetermined time period is calculated by detecting the heartbeat signal (bionic signal) of the measured person 100, the heartbeat information signal (bionic information signal) including the heartbeat information (bionic information) and the transmission interval information is transmitted by the plurality of transmission intervals, on the side of the receiver 201, in accordance with the state of the operating switch 103 or a power source voltage or the like of the battery, the receiving interval is set to the interval in synchronism with either of the transmission intervals to receive the heartbeat information signal (bionic information signal).

Therefore, similar to the first embodiment, when the heartbeat information signal (bionic information signal) from the transmitter 101 is received by the receiver 201, the receiving operation of the receiver 201 can be carried out intermittently, and therefore, the side of the receiver 201 can be constituted by low power consumption formation. Therefore, a heartbeat measuring system (bionic information measuring system) capable of constituting the side of the receiver 201 by low power consumption formation can be provided. Further, the transmitter 101 and the receiver 201 suitable for constructing the heartbeat measuring system (bionic information measuring system) can be provided.

Further, the receiver 201 sets the reception interval by the maximum interval when the voltage of the power source 104 is reduced to be equal to or smaller than the predetermined value even in any state of other parameter for setting the reception interval of the switch 103 or the like. In this way, in the plurality of parameters for setting the reception interval, the state of the power source 104 is constituted by the most predominant parameter, and therefore, even when a remaining amount of power of the power source 104 on the side of the receiver 201 is reduced, the power source 104 can be used longer.

Further, although according to the embodiment, an explanation has been given of an example of the heartbeat measuring system for measuring the heartbeat, the embodiment is applicable to a bionic information measuring system for measuring various kinds of bionic information of pulse, a step number or the like. Further, the embodiment can be utilized for a portable bionic information measuring system having a constitution in which a measured person carries to use at least one of a transmitter and a receiver.

The invention can be utilized for a portable bionic information measuring system having a constitution in which a user carries to use at least one of a transmitter and a receiver starting from the heartbeat measuring system as well as a bionic information measuring system for measuring bionic information of a pulse, walking or the like. Further, the invention can be utilized as a transmitter and a receiver used for the systems.

What is claimed is:

1. A bionic information measuring system comprising:
a transmitter for detecting a bionic signal of a measured person and transmitting in wireless a bionic information signal including bionic information in correspondence with the bionic signal at predetermined intervals; and
a receiver for carrying out a notice indicating the bionic information included in the bionic information signal received by the receiver;
wherein the transmitter includes sensor means for detecting the bionic signal of the measured person and outputting the bionic information in correspondence therewith, and transmitting means for transmitting a signal constituted by adding transmission interval information indicating a transmission interval of the bionic information to the bionic information as the bionic information signal; and
wherein the receiver includes receiving means for receiving the bionic information signal, controlling means for setting a reception interval of the receiving means based on the transmission interval information included in the bionic information signal, and noticing means for carrying out the notice indicating the bionic information of the measured person based on the bionic information signal received by the receiving means.

2. A bionic information measuring system according to claim 1, wherein the transmitter includes transmission interval setting means for setting the transmission interval of the bionic information signal, and the transmitting means transmits the bionic information signal by the transmission interval set by the transmission interval setting means.

3. A bionic information measuring system according to claim 2, wherein the transmitter is constituted by including at least one of operating means capable of setting the transmission interval by an external operation and transmitter power source state detecting means for detecting a state of a power source for the transmitter and setting the transmission interval in accordance with the state of the power source as the transmission interval setting means.

4. A bionic information measuring system according to claim 3, wherein in a case of including at least the transmitter power source state detecting means as the transmission interval setting means, when the transmitter power source state detecting means detects that the power source of the transmitter is reduced to be equal to or smaller than a predetermined voltage and sets the transmission interval, the transmission interval is set the most predominantly by the transmitter power source state detecting means.

5. A bionic information measuring system according to claim 1, wherein the transmitting means of the transmitter transmits the bionic information signal by a plurality of kinds of the transmission intervals;
wherein the receiver includes reception interval setting means for setting a reception interval of the receiving means, the receiving means receives the bionic information signal transmitted by the transmission interval in synchronism with the reception interval set by the reception interval setting means, and the noticing means carries out the notice indicating the bionic information of the measured person based on the received bionic information signal.

6. A bionic information measuring system according to claim 5, wherein the reception interval setting means is constituted by including at least one of operating means capable of setting the reception interval, the receiver power source state detecting means for detecting a state of a power source of the receiver and setting the reception interval in accordance with the state of the power source, and mode detecting means for detecting an operation mode of counting means provided on a side of the receiver and setting the reception interval in accordance with the operation mode of the counting means.

7. A bionic information measuring system according to claim 6, wherein in a case of including at least the receiver power source state detecting means as the reception interval setting means, when the receiver power source state detecting means detects that the power source for the receiver is reduced to be equal to or smaller than a predetermined voltage, the reception interval is set the most predominantly by the receiver power source state detecting means.

8. A transmitter for measuring bionic information comprising:
sensor means for detecting a bionic signal of a measured person and outputting a bionic information in correspondence therewith; and
transmitting means for transmitting a signal constituted by adding transmission interval information indicating a transmission interval of the bionic information to the bionic information as a bionic information signal;
wherein the transmitter for measuring bionic information detects the bionic signal of the measured person and transmits the bionic information signal including bionic information in correspondence with the bionic signal at predetermined intervals.

9. A transmitter for measuring bionic information according to claim 8, further including transmission interval setting means for setting a transmission interval of the bionic information signal, wherein the transmitting means transmits the bionic information signal by the transmission interval set by the transmission interval setting means.

10. A transmitter for measuring bionic information according to claim 9, wherein the transmitter is constituted by including at least one of operating means capable of setting the transmission interval by an external operation and transmitter power source state detecting means for detecting a state of a power source for the transmitter and setting the transmission interval in accordance with the state of the power source.

11. A transmitter for measuring bionic information according to claim 10, wherein in a case of including at least the transmitter power source state detecting means as the transmission interval setting means, when the transmitter power source state detecting means detects that the power source for the transmitter is reduced to be equal to or smaller than a predetermined voltage and sets the transmission interval, the transmission interval is set the most predominantly by the transmitter power source state detecting means.

12. A transmitter for measuring bionic information according to claim 8, wherein the transmitting means transmits the bionic information signal by a plurality of kinds of the transmission intervals.

13. A transmitter for measuring bionic information according to claim 9, wherein the transmitting means transmits the bionic information signal by a plurality of kinds of the transmission intervals.

14. A transmitter for measuring bionic information according to claim 10, wherein the transmitting means transmits the bionic information signal by a plurality of kinds of the transmission intervals.

15. A transmitter for measuring bionic information according to claim 11, wherein the transmitting means transmits the bionic information signal by a plurality of kinds of the transmission intervals.

16. A receiver for measuring bionic information comprising:
a receiver including receiving means for receiving a bionic information signal;
controlling means for setting a reception interval of the receiving means based on transmission interval information included in the bionic information signal; and
noticing means for carrying out a notice indicating the bionic information of a measured person based on the bionic information signal received by the receiving means;
wherein the receiver for measuring bionic information receives the bionic information signal including bionic information of the measured person and transmission interval information indicating a transmission interval of the bionic information and carries out a notice indicating the bionic information of the measured person.

17. A receiver for measuring bionic information according to claim 16, further including reception interval setting means for setting the reception interval of the receiving means, wherein the receiving means receives the bionic information signal transmitted by the transmission interval in synchronism with the reception interval set by the reception interval setting means, and the noticing means carries out the notice indicating the bionic information of the measured person based on the received bionic information signal.

18. A receiver for measuring bionic information according to claim 17, wherein the reception interval setting means is constituted by including at least one of operating means capable of setting the reception interval, receiver power source state detecting means for detecting a state of a power source for the receiver and setting the reception interval in accordance with the state of the power source, and mode detecting means for detecting an operation mode of counting means provided on a side of the receiver and setting the reception interval in accordance with the operation mode of the counting means.

19. A receiver for measuring bionic information according to claim 18, wherein in a case of including at least the receiver power source state detecting means as the reception interval setting means, when the receiver power source state detecting means detects that the power source for the receiver is reduced to be equal to or smaller than a predetermined voltage and the reception interval is set, the reception interval is set the most predominantly by the receiver power source state detecting means.
